(19) United States
(12) Patent Application Publication
(10) Pub. No.: US 2024/0209347 A1
(43) Pub. Date: Jun. 27, 2024

(54) METHOD FOR SIMULTANEOUSLY ISOLATING NUCLEIC ACIDS FROM SEDIMENTABLE AND NONSEDIMENTABLE BIOMOLECULES IN WATER SAMPLES

(71) Applicant: IST INNUSCREEN GMBH, Berlin (DE)

(72) Inventor: Timo HILLEBRAND, Hoppegarten (DE)

(73) Assignee: IST INNUSCREEN GMBH, Berlin (DE)

(21) Appl. No.: 18/547,303

(22) PCT Filed: Feb. 23, 2022
(86) PCT No.: PCT/EP2022/054561

§ 371 Date: Mar. 8, 2024

(30) Foreign Application Priority Data
Feb. 23, 2021 (DE) 10 2021 104 319.0

(51) Int. Cl. C12N 15/10; C12Q 1/6806

(52) U.S. Cl. CPC C12N 15/1006; C12Q 1/6806

(57) ABSTRACT

The invention relates to a simple and very rapid method for simultaneously isolating nucleic acids from sedimentable and nonsedimentable biomolecules in water samples. The water samples can be, inter alia, drinking water, industrial water, surface water or else wastewater. The biomolecules are bacteria, bacteriophages, protozoa, viruses, and free-circulating nucleic acids (e.g., plasmid DNA) encoding antibiotic resistances. According to the invention, a polysaccharide derivative is added to the water sample and the biomolecules am thus enriched. After dissolution of the pellet and lysis of the biomolecules, the nucleic acids are bound to a solid phase, purified and eluted in a known manner.

The invention relates to a simple and very rapid method for simultaneously isolating nucleic acids from sedimentable and nonsedimentable biomolecules in water samples. The water samples can be, inter alia, drinking water, industrial water, surface water or else waste water. The biomolecules are bacteria, bacteriophages, protozoa, viruses and free circulating nucleic acids (for example plasmid DNA) encoding antibiotic resistances.

### STATE OF THE ART

The analysis of all types of water samples using molecular biological methods is is becoming increasingly important. The SARS-CoV-2 pandemic, for example, shows that the analysis of waste water from sewage treatment plants can be significant with regard to an early epidemiological detection of increasing numbers of infections. In addition to a general testing of water for microorganisms, there is an increasing focus on the detection of antibiotic resistances. These resistances are often not associated with bacteria, but are present as freely circulating plasmids. The isolation of nucleic acids from water/waste water samples, however, involves several problems. If a volume of 200 μl is sufficient when testing blood samples for the presence of a viral infection, the sample volume is significantly larger when testing water/waste water. But sample volumes of approx. 20 ml can no longer be used directly for simple and rapid nucleic acid extraction.

But it is also problematic in this context that waste water or industrial process water, for example, contains a large amount of solids and matrix. Surface water from lakes or rivers also contains a relatively high concentration of solids. To reduce or remove these substances from the sample in order to use only the liquid phase for analysis, solid substances can be removed by centrifugation. Other options for removing these substances are filtration techniques, the use of charged filters or else flocculation methods. This reveals a major problem which severely impairs the sensitivity of the analysis methods. It is known that biomolecules bind to solids in water. This means that these proportions of biomolecules to be detected are lost via the sediment if it was removed from the sample before extraction and if only the resulting supernatant is processed. On the other hand, often only the sediment is processed and not the supernatant. Depending on which variant is used, biomolecules are always lost from the respective fraction which is not used. A method which isolates nucleic acids from both fractions simultaneously is not known.

It should be noted here that there is a number of techniques for concentrating biomolecules from a large-volume sample.

For example, ultracentrifugation techniques can be used to enrich viruses or subcellular particles from a biological sample. Moreover, also the ultrafiltration technique is being used. This method as well is time-consuming and very expensive. Alternative methods consist of the precipitation of virus particles using polyethylene glycol/sodium chloride and subsequent centrifugation (Yamamoto et al., Virology 40(1970) 734; Morandi et al., J. Clin. Microbiol. 36 (1998) 1543-1538). Various mixtures of PEG and sodium chloride are used here and these reagents are mixed with the biological sample. The preparation is then incubated at low temperatures for a longer period of time and the virus (protein) NaCl/PEG precipitates are subsequently obtained by centrifugation. These methods as well are complex and take a lot of time. Another problem is further processing of the precipitates for the isolation of the viral nucleic acids. The precipitates are often very difficult to dissolve again. This has a significant impact on the efficiency and quality of nucleic acid isolation. The patent specification DE 19856415 C2 describes a method which makes use of the known NaCl/PEG precipitation, wherein the isolation of the nucleic acids is subsequently realized in a known manner by binding to a siliceous solid phase. It is not clear to what extent this method differs from the well-known method of NaCl/PEG precipitation with its well-known problems. In addition, this method also requires cold incubation and centrifugation for twenty minutes. The method should make it possible to isolate viral nucleic acids from a sample of up to 10 ml. Ultimately, all these method are very time-consuming and labor-intensive and also require special and expensive equipment (e.g. ultracentrifuge).

### OBJECT OF THE INVENTION

The object of the present invention was to eliminate the disadvantages of the known technical solutions. The aim of the present invention is therefore to provide a simple and rapid method which allows the simultaneous isolation of nucleic acids from a wide variety of biomolecules present in water samples.

### Solution

The problem was solved in accordance with the features of the patent claims. The simultaneous and parallel-simultaneous isolation according to the invention was carried out from a sediment obtained after centrifugation and from the liquid phase formed after centrifugation. The sediment and liquid phase are separated by simple centrifugation. The method ensures that there is no loss of nucleic acids due to the simultaneous processing of sediment and the resulting liquid phase, thus significantly increasing the sensitivity of molecular genetic detection methods.

In addition, the method also allows the simultaneous parallel extraction of nucleic acids from biomolecules which are per se only in the sediment (for example bacteria), from biomolecules that are mainly in the liquid phase after centrifugation (for example, circulating free DNA, bacteriophages or viruses).

The problem is surprisingly easily solved by the present invention. Commercially available extraction reagents can be used here. The decisive factor is the combination of these to enable the task to be solved. The simultaneous isolation of nucleic acids from sediment and the resulting supernatant is solved according to the invention as follows: 20 ml of a water is sample. e.g. waste water from a sewage treatment plant, are transferred to a 50 ml centrifugation vessel. The sample is centrifuged at 5,000 rpm for 20 minutes to separate the sediment from the supernatant and to obtain a sediment-free liquid phase. The supernatant is completely removed of which 10 ml are transferred to a new centrifugation vessel for further processing. The sediment is resuspended with 1×PBS buffer and transferred to a new 1.5 ml centrifugation vessel. Both fractions are now available for simultaneous extraction of the nucleic acids. The supernatant now contains the biomolecules that cannot be sedimented by simple centrifugation (free nucleic acid, bacteriophages, viruses, etc.). The processing of the supernatant (10 ml) with the aim of concentrating the biomolecules contained is based on the use of polysaccharide derivatives for complexing the biomolecules contained in a sample. The polysaccharide derivatives are the salts of polyuronic acids. The so-called alginates of alginic acids are particularly suitable. Alginates are elements providing structure in brown algae. Alginate is a polysaccharide consisting of 1,4-linked α-L-guluronic acid (G) and β-D-mannuronic acid (M). This is disclosed in the published patent application DE 102008023297 A1 (also in WO2009/135936A1 and in US020110117628A1). These publications describe the 0.0 use of the ability of alginates to gel in solutions with a low calcium content and to form so-called hydrogels. Gelation is caused by the incorporation of calcium ions into the zigzag structure of the GG blocks. The zigzag structure of another alginate molecule is then deposited on this zone. This results in the formation of three-dimensional structures. The formation of gels also occurs in combination with strong acids. The resulting gel structures can also be specifically destroyed again. But no conclusions can be drawn as to the application of this technical solution in a method for sedimentable and nonsedimentable biomolecules in water samples, as it remains unclear whether these methods only enrich the biomolecules from the liquid phase or also from the sediment phase with gel formation. This has been described in table 1 (paragraphs [0030] and [0031]).

By utilizing the formation of alginate gels, the enrichment of biomolecules (viruses, bacteriophages, free nucleic acids, etc.) can be carried out easily and quickly, followed by the isolation of nucleic acids.

An alginate solution and a calcium chloride solution are therefore added to the liquid phase (10 ml supernatant). The preparation is briefly shaken, incubated and then centrifuged for 20 minutes at 5,000 rpm. The supernatant is removed and the process is continued with the pellet. This step makes it possible to concentrate the volume of the sample from 10 ml to a few microliters.

A commercially available lysis buffer (Lysis Solution RL; Analytik Jena AG) is added is to the pellet obtained after concentration. The pellet is dissolved using the lysis buffer, which means that the biomolecules of the liquid phase are now present in the lysis buffer. The sediment fraction resuspended in the PBS buffer is processed as follows:

Depending on the biomolecules in the sediment from which the nucleic acids are to be extracted (for example, from viruses or free nucleic acids which were bound to sediment components or from bacteria which were also part of the sediment due to the previous centrifugation), the sediment fraction is briefly centrifuged and the supernatant is further used directly or the resuspended sediment is lysed enzymatically or thermally in advance, then centrifuged and the supernatant is used for further extraction after this lysis. This is added to the vessel containing the lysis buffer from the liquid phase. Now all the biomolecules from the liquid phase and the sediment fraction are under lysis buffer in a reaction vessel.

The nucleic acids can now be extracted using methods known to the person skilled in the art. For this purpose, after a short lysis step, the nucleic acid is bound to a solid phase (for example, to a spin filter containing a glass fiber membrane or to magnetic or paramagnetic particles) with the addition of a binding buffer. This is followed by a washing step, and the nucleic acid is finally separated from the solid phase after the addition of water or a low salt buffer. The present nucleic acids consist additively of the biomolecules of the sediment fraction and the supernatant fraction which were obtained in the first steps from a 20 ml waste water sample. The entire procedure takes just over an hour. It can be carried out both manually and automatically. It does not require ultracentrifugation or ultrafiltration techniques which are usually used for processing water samples. The method enables the extraction of nucleic acids from all biomolecules contained in the sample.

The method of parallel extraction of biomolecules from both fractions can also still be simplified. This can be advantageous if the sample volume is smaller (preferably less than 15 ml). In the process, the sample is not even separated into a sediment fraction and a liquid phase. An alginate solution and a calcium chloride solution are added to the entire sample (for example, 10 ml). The preparation is shaken briefly, incubated and then centrifuged for 20 minutes at 5,000 rpm. The supernatant is removed and the process continues with the pellet. The pellet now contains the sediment and the concentrated biomolecules of the liquid phase. A commercially available lysis buffer (Lysis Solution RL; Analytik Jena AG) and proteinase K are again added to the pellet, it is transferred to a 1.5 ml reaction vessel and subsequently incubated for 20 minutes at 60° C. The preparation is then centrifuged at maximum speed for a few minutes and the supernatant is transferred to a new reaction vessel. The extraction of nucleic acids can now be carried out again using known methods. For this purpose, after a is short lysis step, the nucleic acid is bound to a solid phase (e.g. to a spin filter containing a glass fiber membrane or to magnetic or paramagnetic particles) with the addition of a binding buffer. This is followed by a washing step, and the nucleic acid is finally separated from the solid phase after the addition of water or a low salt buffer. The present nucleic acids consist additively of the biomolecules of the sediment fraction and the supernatant fraction which were obtained in the first steps from a 10 ml waste water sample. This procedure can be carried out even faster, as it does not require the separation of sediment and liquid phase.

In addition to simultaneous extraction, the method can also be ideally used for simultaneous parallel isolation of nucleic acids from sediment and the resulting supernatant. Only the sediment fraction and the supernatant fraction are not combined here. Both fractions are each processed in parallel as separate samples. The nucleic acids obtained are then the nucleic acids from the respective fraction.

The method according to the invention for the simultaneous or parallel simultaneous isolation of nucleic acids from a mixture of sedimentable and nonsedimentable biomolecules in water samples is characterized by the following steps:
a) Separating the sample into a sediment fraction and a liquid phase. A centrifuge is preferably used for this. The nonsedimentable phase (supernatant) contains, among other things, the free nucleic acids and cells which have not been sedimented.
b) Due to the large volume, which is unsuitable for nucleic acid isolation, the non-sedimentable biomolecules of the liquid phase are enriched in a pellet by adding a polysaccharide derivative, preferably an alginate. Preferably, a calcium salt is added to the liquid phase and all the cells and biomolecules of the liquid phase are now in this pellet.
c) Dissolving the pellet by adding a lysis buffer. This not only dissolves the pellet, but also lyses any cells present.
d) Lysis of the sediment fraction and transfer of the lysed components to another liquid phase. This lysis releases the nucleic acids of the components of the sedimented phase.
e) Either combining the two liquid phases and binding the nucleic acids to a solid phase or separately binding the nucleic acids of the two liquid phases and purifying and eluting them using known methods.

A simple variant of the method according to the invention for simultaneous isolation of nucleic acids from a mixture of sedimentable and nonsedimentable biomolecules in water samples is characterized by the following steps:
a) Enrichment of the nonsedimentable biomolecules of the liquid phase in a pellet by adding a polysaccharide derivative without separating the sediment
b) Dissolving the pellet by adding a lysis buffer and simultaneously lysing the sediment fraction and transferring the lysed components to another liquid phase
c) Binding of the nucleic acids of this liquid phase to a solid phase and their purification and elution according to known methods.

The separation into a liquid and sediment phase is omitted. This variant has its advantages for volumes up to 15 ml.

The lysis, the binding of the nucleic acids released by lysis and the processing are known to the person skilled in the art. One possible method is magnetic separation. But it would also be possible to bind the nucleic acids to a rough surface (according to WO 2016/169677 A1).

The use of the method according to the invention is the determination of the total content of nucleic acids from a mixture of sedimentable and nonsedimentable biomolecules in water samples. This determination corresponds to a simultaneous isolation of the nucleic acids. Another use according to the invention is the determination of the content of nucleic acids of the sedimentable and/or nonsedimentable components from a mixture of sedimentable and nonsedimentable biomolecules in water samples.

The invention is explained in more detail below with reference to embodiments, wherein the embodiments do not imply any limitation of the method according to the invention.

### EMBODIMENTS

### Example 1: Simultaneous Extraction and Simultaneous Parallel Extraction of Nucleic Acids from Bacteria Contained in a Water Sample and Free Bacteriophage RNA

Surface river water from the Havel was taken as the initial samples. The samples were spiked with bacteria (salmonella) and a bacteriophage RNA (MS2-RNA).

For nucleic acid extraction, the samples were separated into a sediment fraction and a liquid phase. Always 20 ml of the initial sample were used. It can be assumed that the bacteria are in the sediment and the free RNA is in the liquid phase as non-sedimentable biomolecules, but that, if applicable, a proportion of the free nucleic acid may also bind to suspended matter in the sample and is therefore part of the sediment. The aim is to isolate both the bacterial nucleic acid and the free nucleic acid quickly and easily. The simultaneous extraction of nucleic acids from both fractions was intended to achieve the maximum possible yield. In addition to the simultaneous extraction from both fractions, a simultaneous parallel extraction is from the sediment and the liquid phase was also carried out to show which biomolecules were present in which of the fractions.

The extraction was carried out according to the method of the invention as follows. 20 ml of the sample were centrifuged in a 50 ml reaction vessel for 20 minutes at 5,000 rpm. The supernatant was removed and 10 ml of it were transferred to a new vessel.

1. Simultaneous Parallel Extraction of Nucleic Acids from Sediment and Liquid Phase
1.1. Extraction of Nucleic Acid from the Liquid Phase

100 μl alginate solution and 100 μl calcium chloride solution were added to the 10 ml liquid phase. The preparation was briefly shaken, incubated for 10 minutes and then centrifuged at 5,000 rpm for 20 minutes. After this centrifugation, the supernatant was removed and the pellet was mixed with 500 μl of a lysis buffer (Lysis Solution RL; Analytik Jena) and the pellet was dissolved by pipetting several times, transferred to a new 1.5 ml reaction vessel and mixed with 20 μl proteinase K and incubated at 60° C. for 15 minutes. 50 μl of a magnetic particle solution and 450 μl of a binding buffer (Binding Solution SBS; Analytik Jena) were added to the sample. The preparation was briefly mixed and incubated for 2 minutes. The magnetic particles were then separated using a magnet. The supernatant was removed and the magnetic particles were washed with 1 ml of a wash buffer (Washing Solution HS; Analytik Jena). After separating the magnetic particles again, the supernatant was completely removed and the magnetic particles were washed two more times with 80% ethanol. The reaction vessel was then incubated with the lid open in a thermomixer at 50° C. for 10 minutes. Elution was performed by adding 50 μl RNAse free water and incubating at 60° C. for 5 minutes. After separation of the magnetic particles, the supernatant was removed and transferred to a new vessel. The vessel now contains only the nucleic acid from the liquid phase.

1.2. Extraction of Nucleic Acid from the Sediment

The sediment pellet obtained after centrifugation was resuspended in 250 μl 1 PBS buffer and incubated at 37° C. for 30 minutes after the addition of 10 ml lysozyme. The sediment sample was then centrifuged for 2 minutes at 12,000 rpm and the supernatant was transferred to a new reaction vessel and mixed with 300 μl of a lysis buffer (Lysis Solution RL; Analytik Jena) and 20 μl proteinase K and incubated at 60° C. for 15 minutes. 50 μl of a magnetic particle solution and 450 μl of Binding Solution SBS (Analytik Jena) were added to is the sample. The preparation was briefly mixed and incubated for 2 minutes. The magnetic particles were then separated using a magnet. The supernatant was removed and the magnetic particles were washed with 1 ml of a wash buffer (Washing Solution HS; Analytik Jena). After separating the magnetic particles again, the supernatant was completely removed and the magnetic particles were washed two more times with 80% ethanol. Subsequently, the reaction vessel was incubated with the lid open in a thermomixer at 50° C. for 10 minutes. Elution was performed by adding 50 μl of RNAse free water and incubating at 60° C. for 5 minutes. After separation of the magnetic particles, the supernatant was removed and transferred to a new vessel. The vessel now contains only the nucleic acid from the sediment.

2. Simultaneous extraction of nucleic acids from sediment and liquid phase

The sediment pellet obtained after centrifugation was resuspended in 250 μl 1 PBS buffer and incubated at 37° C. for 30 minutes after the addition of 10 ml lysozyme. During the incubation period, 100 μl of alginate solution and 100 μl of calcium chloride were added to the 10 ml of liquid phase. The preparation was briefly shaken, incubated for 10 minutes and then centrifuged at 5,000 rpm for 20 minutes. After this centrifugation, the supernatant was removed and the pellet was mixed with 300 μl Lysis Solution RL (Analytik Jena) and the pellet was dissolved by pipetting several times and transferred to a new 1.5 ml reaction vessel. After lysis of the sediment sample, it was centrifuged for 2 minutes at 12,000 rpm and the supernatant was then added to the liquid phase sample already present under Lysis Solution RL. Both samples are thus combined for further processing. After addition of proteinase K, this sample was incubated at 60° C. for another 15 minutes. 50 μl of a magnetic particle solution and 450 μl of Binding Solution SBS (Analytik Jena) were added to the sample. The preparation was briefly mixed and incubated for 2 minutes. The magnetic particles were then separated using a magnet. The supernatant was removed and the magnetic particles were washed with 1 ml of a wash buffer (Washing Solution HS: Analytik Jena). After separating the magnetic particles again, the supernatant was completely removed and the magnetic particles were washed two more times with 80% ethanol. The reaction vessel was then incubated with the lid open in a thermomixer at 50° C. for 10 minutes. Elution was performed by adding 50 μl of RNAse free water and incubating at 60° C. for 5 minutes. After separation of the magnetic particles, the supernatant was removed and transferred to a new vessel. The vessel now contains nucleic acid from the sediment as well as from the liquid phase.

In order to check in which of the fractions (sediment and liquid phase) the bacteria and the free bacteriophage RNA were located, the nucleic acids obtained from each extraction variant performed were tested using real-time PCR. Salmonella DNA and bacteriophage RNA are detected using specific assays. The CT values after real-time PCR are listed in the table below.

TABLE 1

| Sample | Salmonella | Bacteriophage RNA |
|---|---|---|
| Sample 1 (sediment) | 19.5 | 27.5 |
| Sample 2 (sediment) | 19.5 | 27.6 |
| Sample 1 (liquid phase) | not detectable | 26.0 |
| Sample 2 (liquid phase) | not detectable | 25.9 |
| Sample 3 (sediment + liquid phase) | 19.5 | 25.4 |
| Sample 4 (sediment + liquid phase) | 19.6 | 25.2 |

The data confirm the previous assumption that the bacteria are located in the sediment and that the free bacteriophage RNA is primarily located in the liquid phase, but also in portions in the sediment (approx. 70% in the liquid phase and 30% in the sediment).

The results show impressively that with only one fraction for the extraction of nucleic acids from the bacteria and/or the free bacteriophage RNA, no nucleic acid would be obtained and/or the yield would be insufficient. The method of simultaneous isolation captures all targets to be isolated quickly and easily, thus allowing the maximum yield of nucleic acid to be obtained.

### Embodiment 2: Simultaneous Extraction of Nucleic Acids from Bacteria Contained in a Water Sample as Well as Free Bacteriophage RNA and Free Plasmid DNA

Heavily contaminated water from a fire extinguishing pond was taken as initial samples. Bacteria (Salmonella), synthetic plasmid DNA and bacteriophage RNA (MS2-RNA) were added to the samples.

For nucleic acid extraction, the samples were separated into a sediment fraction and a liquid phase. Always 20 ml of the initial sample were used. It can be assumed that the bacteria to are in the sediment and the free RNA and the free plasmid DNA are in the liquid phase as nonsedimentable biomolecules, but, where applicable, also a proportion of the free nucleic acid binds to suspended matter in the sample and is therefore part of the sediment. The aim is to isolate both the bacterial nucleic acid and the free nucleic acids quickly and easily. In this embodiment, the nucleic acids are not bound to magnetic particles, but to a spin filter with a is glass fiber membrane.

The extraction was carried out according to the method of the invention as follows. 20 ml of the sample were centrifuged in a 50 ml reaction vessel for 20 minutes at 5,000 rpm. The supernatant was removed and 10 ml transferred to a new vessel.

The sediment pellet obtained after centrifugation was resuspended in 250 μl 1 PBS buffer and incubated at 37° C. for 30 minutes after addition of 10 ml lysozyme. During the incubation period, 100 μl of alginate solution and 100 μl of calcium chloride were added to the 10 ml of liquid phase. The preparation was briefly shaken, incubated for 10 minutes and then centrifuged at 5,000 rpm for 20 minutes. After this centrifugation, the supernatant was removed, the pellet was mixed with 300 μl Lysis Solution RL (Analytik Jena), dissolved by pipetting several times and transferred to a new 1.5 ml reaction vessel. After lysis of the sediment sample, the same was centrifuged for 2 minutes at 12,000 rpm and the supernatant was then added to the liquid phase sample already present under Lysis Solution RL. Thus, both samples are combined for further processing. After the addition of proteinase K, this sample was incubated at 60° C. for a another 15 minutes. 450 μl Binding Solution SBS (Analytik Jena) was added to the sample. The preparation was briefly mixed, placed on a Spin Filter column and centrifuged for 1 minute at 11,000×g. The filtrate was discarded. The Spin Filter column was subsequently washed twice with 600 μl of a wash buffer (Washing Solution HS; Analytik Jena) and/or twice with 80% ethanol. After the last washing step, the Spin Filter column was centrifuged for 3 minutes at maximum speed and then the Spin Filter column was placed in an empty reaction vessel. 100 μl of RNAse-free water were then added to the Spin Filter column and subsequently centrifuged at 11,000×g for one minute. The vessel now contains nucleic acid from the sediment as well as from the liquid phase. According to the invention, the suspected nucleic acids could be isolated easily and quickly from both fractions.

The isolated bacterial DNA, plasmid DNA and bacteriophage RNA were detected using real-time PCR The CT values after real-time PCR are listed in the table below.

TABLE 2

| Sample | Salmonella | Bakteriophage RNA | Plasmid-DNA |
|---|---|---|---|
| Sample 1 (sediment + liquid-phase) | 25.0 | 22.5 | 24.5 |
| Sample 2 (sediment + liquid-phase) | 24.8 | 22.2 | 24.4 |

### Embodiment 3: Simultaneous Extraction of Nucleic Acids from Bacteria Contained in a Water Sample as Well as Free Bacteriophage RNA and Free Plasmid DNA without Prior Separation of Sediment and Liquid Phase

Heavily contaminated water from a fire extinguishing pond was taken as initial is samples. Bacteria (Salmonella), synthetic plasmid DNA and bacteriophage RNA (MS2-RNA) were added to the samples.

A 10 ml sample was used. The sample was transferred to a 15 ml reaction vessel and 100 μl alginate solution and 100 μl calcium chloride were added. The preparation was briefly shaken, incubated for 10 minutes and then centrifuged at 5,000 rpm for 20 minutes. After this centrifugation, the supernatant was removed, the pellet was mixed with 500 μl Lysis Solution RL (Analytik Jena), dissolved by pipetting several times and transferred to a new 1.5 ml reaction vessel and mixed with 20 μl of a proteinase K solution. After incubation at 60° C. for 20 minutes, the preparation was centrifuged for 3 minutes at 14,000 rpm and the supernatant was transferred to a new 1.5 ml reaction vessel. 200 μl of a binding buffer (Binding Solution SBS; Analytik Jena) were then added to the sample. The preparation was briefly mixed, placed on a Spin Filter column and centrifuged for 1 minute at 11,000×g. The filtrate was discarded. The Spin Filter column was subsequently washed twice with 600 μl of a wash buffer (Washing Solution HS; Analytik Jena) an/or twice with 80% ethanol. After the last washing step, the Spin Filter column was centrifuged for 3 minutes at maximum speed and then the Spin Filter column was put in an empty reaction vessel. 100 μl of RNAse-free water were then added to the Spin Filter column and subsequently centrifuged at 11,000×g for one minute. The vessel now contains nucleic acid from the sediment as well as from the liquid phase. According to the invention, the suspected nucleic acids could be isolated easily and quickly from both fractions without having previously separated the two fractions.

The isolated bacterial DNA, plasmid DNA and bacteriophage RNA were detected using real-time PCR. The CT values after real-time PCR are listed in the table below.

TABLE 3

| Sample | Salmonella | Bakteriophage RNA | Plasmid-DNA |
|---|---|---|---|
| Sample 1 (sediment + liquid-phase; | 23.7 | 25.5 | 26.5 |
| without separation) | | | |
| Sample 2 (sediment + liquid-phase; | 23.8 | 26.2 | 27.3 |
| without separation) | | | |

1. A method for simultaneously or parallel-simultaneously isolating nucleic acids from a mixture of sedimentable and nonsedimentable biomolecules in water samples, characterized by the following steps:
a) separating the sample into a sediment fraction and a liquid phase
b) enriching the nonsedimentable biomolecules of the liquid phase in a pellet by adding a polysaccharide derivative
c) dissolving the pellet by adding a lysis buffer
d) lysis of the sediment fraction and transfer of the lysed components into another liquid phase
e) either combination of both liquid phases and binding of the nucleic acids to a solid phase or separate binding of the nucleic acids of the two liquid phases as well as their purification and elution according to known methods.

2. The method for simultaneous isolation of nucleic acids from a mixture of sedimentable and nonsedimentable biomolecules in water samples, characterized by the following steps:
d) enriching the nonsedimentable biomolecules of the liquid phase in a pellet by adding a polysaccharide derivative without separating the sediment
e) dissolving the pellet by adding a lysis buffer and simultaneous lysis of the sediment fraction and transfer of the lysed components to another liquid phase
f) binding the nucleic acids of said liquid phase to a solid phase and their purification and elution according to known method.

3. The method according to patent claim 1 or 2, characterized in that alginates are used as polysaccharide derivative.

4. The method according to patent claim 1 or 2 and 3, characterized in that a salt of a double positively charged cation, preferably a calcium salt, is added to the water sample or the solid phase.

5. A use of the method according to any one of patent claims 1 to 4 for determination of the total content of nucleic acids from a mixture of sedimentable and nonsedimentable biomolecules in water samples.

6. The use of the method according to any one of patent claim 1 or 3 and 4 for determination of the content of nucleic acids of the sedimentable components from a mixture of sedimentable and nonsedimentable biomolecules in water samples.

7. The use of the method according to any one of patent claim 1 or 3 and 4 for determination of the content of nonsedimentable components from a mixture of sedimentable and nonsedimentable biomolecules in water samples.
